(19) United States
(12) Patent Application Publication
(10) Pub. No.: US 2023/0049642 A1
(43) Pub. Date: Feb. 16, 2023

(54) DEVICE AGNOSTIC SLEEP STAGING

(71) Applicant: CERNO HEALTH INC., San Carlos, CA (US)

(72) Inventors: Anusha Viddam, San Carlos, CA (US); Hedyeh Agheh Kholerdi, Redwood City, CA (US); Srikanth Tummala, Collegeville, PA (US)

(21) Appl. No.: 17/885,940

(22) Filed: Aug. 11, 2022

Related U.S. Application Data
(60) Provisional application No. 63/232,906, filed on Aug. 13, 2021.

(51) Int. Cl. G16H 50/20

(52) U.S. Cl. CPC G16H 50/20

(57) ABSTRACT

The invention provides systems and methods that stage sleep in patients using wearable devices regardless of the brand or features of the wearable device. Data collected from wearable devices are assigned a sleep stage regardless of manufacture or mode of operation. Methods provide precise and accurate sleep stage information to physicians via an online portal of the system, offering patients suffering from poor sleep opportunities for better medical outcomes. Methods include preprocessing data for a subject from a first sensor on a first wearable device; based on a type of the wearable device into standardized data and analyzing the standardized to identify sleep stages. Second data from a second wearable device with different formats or different content is also preprocessed a format matching that of the standardized data.

### TECHNICAL FIELD

The disclosure relates to sleep health and medical assessment of sleep.

### BACKGROUND

Up to 70 million Americans suffer from sleep or wakefulness disorders. Poor sleep is a significant health problem that may manifest as impaired cognitive performance, cardiovascular disease, clinical depression, or an overall poor quality of life. Poor sleep quality has also been implicated in medical conditions such as stroke, diabetes, Alzheimer’s disease, and mental health. Sleep deprivation also comes with a high social cost, causing increased mortality, diminished societal engagement, and poor educational outcomes. Poor sleep is likely a product of a complex interplay of a diversity of different contributing factors, inborn, nurtured, and environmental.

To clinically assess sleep, a sleep clinician may refer a patient for polysomnography (PSG), an in-patient procedure that uses connected electrodes to record brain waves, oxygen levels, and eye and leg movements from a patient for one night. The PSG visit typically has the patient sleep for the night in a hospital with sensors attached to his or her scalp, face, and legs. Unfortunately, the one night in a hospital may not represent what the patient experiences over months or years in their own bed.

Given the obtrusive nature of a polysomnography, only the most progressed patients get the polysomnography.

### SUMMARY

There is a need for widely accessible novel technology that can aid in the diagnosis and management of sleep disorders. The invention provides systems and methods that stage sleep in patients using wearable devices such as smartwatches and in which the sleep staging is performed neutrally and reliably regardless of the brand or features of the wearable device used by the patient. Systems and methods of the invention perform sleep staging and provide accurate sleep evaluation measures. Systems and methods of the invention use data that is collected from wearable devices while patient sleep, and assign a sleep stage—such as wake, REM sleep or non-REM (NREM) sleep—to different segments of time, or epochs, throughout the night. Systems and methods use various tools and features to allow patients to use their own various wearable devices, regardless of manufacture or mode of operation. For example, systems of the invention may include software modules that interact specifically with application programming interfaces (APIs) of different devices. Additionally or alternatively, systems of the invention may perform some preprocessing steps on data from the wearable devices so that the data from at least one of the device types is standardized to match data as used throughout the system. For example, the system may use or receive data from devices that measure heart rate and activity, and the system may typically analyze data that includes one heart rate value per second (Hz), where, for example, some common wearable device may measure heart rate at 1 Hz. When the system interacts with a second device type that measures heart rate at another frequency (e.g., once every five seconds), the system may interpolate heart rate measurements from the second device so that records stored within the system from the second device include one heart rate value per second (Hz).

Additionally, systems and methods of the invention may instruct the operation of wearable devices in a manner that allows the system to easily interoperate with a diversity of different wearable devices. For example, where the system prefers a standard heart rate frequency that is higher that a default heart rate sampling frequency of a device, the system can send instructions to the device, via that device’s API, instructing that device to operate in a different, or optional, mode of operation that captures heart rate at a higher frequency than by default. For example, some smartwatches or chest-straps have a default sampling rate and offer their users an optional “workout mode”, intended to allow fitness-minded users to measure heart rate at a high resolution when exercising. Systems of the invention instruct such devices to operate in that optional (e.g., “workout”) mode, capturing heart rate according to a system-wide standard.

The system analyzes the data that are captured. For example, the system may bin a night’s worth of sleep data into short (e.g., 30 second) epochs, and classify each epoch as representing a certain stage of sleep (e.g., wake, REM, NREM). The system can perform the classification using a classifier such as a machine learning system. A machine learning system may be trained on a large training data set (e.g., hundreds of thousands of subjects) labeled with “gold standard” techniques such as PSG data labeled by clinicians. The machine learning system can output results night-after-night showing when a patient slept and in what stages. The system provides a valuable tool by which a physician can diagnose and monitor patient sleep patterns. Because the system provides primarily a medical tool with clinical-grade data, it is not strictly necessary for the system to provide classification results in real-time, minute-by-minute, or to be able to show those results to the patient (e.g., on a screen of a wearable device or via a companion app on a personal device such as a smartphone). While the system may optionally be able to share information in that manner, more importantly, it need only upload data from the wearable devices, through any smartphone apps, to the system periodically, in batches and/or when connectivity is available.

Machine learning systems of the invention make use of the insight that there are naturally-occurring time-dependencies in measurements of the course of a patient’s sleep and wake time. The machine learning system captures and benefits from capturing those time-dependencies through the use of one or more classifiers, e.g., neural networks that capture time dependencies. For example, the machine learning system may include a recurrent neural network or even a long short-term memory (LSTM) neural network. Machine learning systems of the invention have shown to be highly precise and accurate in validation studies, giving physicians a valuable tool in understanding how their patients sleep or what disturbances or irregularities may manifest in patient sleep patterns. Because systems and methods of the invention work agnostically with a variety of different types of wearable devices, including common smartwatches, patients may comfortably avail themselves of system and methods of the invention, capturing the medical data over many nights (and days), at home, and without significant disruption to their regular sleep patterns.

Because the patients may provide sleep-related data at home, over multi-night spans of time, in familiar environments, using simple wearable devices, data in the system are representative of the patients likely, ongoing sleep patterns and experiences. Because the machine learning system uses time dependencies, the physician has access to data with a very high degree of precision and accuracy, reliably showing patient sleep patterns. Because systems and methods of the invention are device agnostic, different patients with different devices may be treated uniformly with comparable results. In fact, a benefit of systems and methods of the invention is that the evaluation, treatment, and monitoring of a patient is uniform and not-disrupted even if that patient changes brand or type of wearable device. Regardless of the device type, or changes of device type, the physician receives highly precise and accurate sleep information which may include sleep stage information from patients over time, other core metrics, or combinations thereof. The sleep stage information may be written by the system to electronic health records or accessed, for example, by the physician via an online portal of the system. Those tools give physicians ability to understand and treat patients suffering from poor sleep, offering those people better medical outcomes and better quality of life.

In certain aspects, the invention provides methods for analyzing sleep patterns. Methods include receiving, at a computer system, data for a subject from a first sensor on a first wearable device; preprocessing the data based on a type of the wearable device into standardized data; and analyzing the standardized data with a computer system to identify sleep stages at different times for the subject. The method may include receiving second data from a second subject using a second wearable device, where the data and the second data are output from the respective devices with different formats or different content. The method may further include preprocessing the second data into a format matching that of the standardized data.

In some embodiments, the first wearable device and the second wearable device each include at least one photoplethysmographic (PPG) sensor and at least one accelerometer. The first wearable device and the second wearable device may output either of the PPG and acceleration data with different formats or sampling frequencies and the method operates uniformly, regardless. The computer system may include one or more software modules that interact with the application programming interfaces (APIs) associated with different wearable devices from different manufacturers. Optionally, the method may include receiving second data from a second subject using a second wearable device, in which the computer system instructs the second wearable device to turn on an optional mode (such as a “workout mode”). In the optional mode, the second device may capture heart rate at a resolution greater than a default mode for the second wearable device.

In preferred embodiments, the analyzing step involves presenting the standardized data to a machine learning system that assigns a sleep stage to each of a plurality of epochs in the standardized data (e.g., 3-stage staging with wake, REM, NREM; 4-stage with wake, REM, NREM deep, NREM light; 5-stage; or others). Preferably the machine learning system includes one or more algorithms that capture time dependencies. Algorithms that capture time dependencies may include recurrent neural such as a long short-term memory neural network. The machine learning system has preferably been trained on training data captured from multiple subjects. The machine learning system preferably outputs records of sleep stages or intervals for each subject, in which contents of the records are consistent regardless of the different formats or different content between the data from the wearable device and the second data from the second wearable device.

In certain embodiments, the computer system creates a record of sleep stages for the patient, and provides access to the record to a clinician who is a registered user of the computer system. The computer system may have stored therein a profile for each of a plurality of patients, each profile including a patient specific circadian model, e.g., with information identifying a resting heart rate or a heart rate variability threshold for a respective patient. The profile may be built over a span of days, e.g., the span may be a rolling period of the prior N (e.g., 7) days, in which cases the profile may change over time—e.g., to generally reflect the patient experience over the prior N days. The analyzing step of the method may include filtering and smoothing the standardized data, comparing the smoothed data to thresholds from the profile for the subject, merging sleep sequences based on the thresholds, and creating a record with sleep intervals for the subject. The sleep interval in the record preferably includes a sequence of bedtime, sleep onsite, wakeup, and rise time. That is, methods of the invention support the recognition that important part of a night of sleep include at least the four specific times when the patient went to bed, went to sleep, woke up, and rose from bed.

Related aspects of the disclosure provide a system for analyzing sleep patterns. The system includes a processor coupled to memory containing instructions executable to cause the system to: receive data for a subject from a first sensor on a first wearable device; preprocess the data based on a type of the wearable device into standardized data; and analyze the standardized data to identify sleep stages at different times for the subject. The system may be operable to receive second data from a second subject using a second wearable device, wherein the data and the second data are output from the respective devices with different formats or different content. Preferably the system is operable to preprocess the second data into a format matching that of the standardized data. The first wearable device and the second wearable device may each include at least one photoplethysmographic (PPG) sensor and at least one accelerometer. The first wearable device and the second wearable device may output PPG and acceleration data with different formats. The system may include one or more software modules that interact with each application programming interface (API) associated with a plurality of different wearable devices from different manufacturers. The system may be operable to receive second data from a second subject using a second wearable device; and instruct the second wearable device to turn on an optional mode (e.g., a workout mode or other) that captures heart rate at a resolution greater than a default mode for the second wearable device.

In preferred embodiments, the system presents the standardized data to a machine learning system that assigns a sleep stage (e.g., 3-stage, 4-stage, etc.) to each of a plurality of epochs in the standardized data. The machine learning system may include at least one neural networks that captures time dependencies (e.g., RNN or LSTM). Preferably, the machine learning system has been trained on labeled training data from multiple subjects. The machine learning system outputs records of sleep stages or intervals for each subject. Contents of the records are preferably consistent regardless of the different formats or different content between the data from the wearable device and the second data from the second wearable device. In preferred embodiments, the system creates a record of sleep stages for the patient, and provides access to the record to a clinician who is a registered user of the computer system. The system may keep, stored therein, a profile for each of a plurality of subjects, each profile identifying a resting heart rate or a heart rate variability threshold for a respective subject. The analyzing step performed by the system may include filtering and smoothing the standardized data, comparing the smoothed data to thresholds from the profile for the subject, merging sleep sequences based on the thresholds, and creating a record with sleep intervals for the subject. System records may include at least one sequence of bedtime, sleep onsite, wakeup, and rise time.

### BRIEF DESCRIPTION OF THE DRAWINGS

FIG. 1 diagrams steps of a method for analyzing sleep patterns.

FIG. 2 is a diagram of a system of the disclosure.

FIG. 3 shows a wearable device and a personal device.

FIG. 4 diagrams preprocessing operations according to certain embodiments.

FIG. 5 diagrams a method of analyzing sleep using a personalized circadian model.

FIG. 6 shows building a circadian model.

FIG. 7 shows use of the circadian model to identify sleep stages.

FIG. 8 shows the steps in sleep staging according to the methods of the disclosure.

FIG. 9 shows preparation of input to a machine learning model.

FIG. 10 shows use of a machine learning system of the disclosure.

FIG. 11 gives one exemplary architecture for a machine learning system.

### DETAILED DESCRIPTION

The disclosure provides systems and methods for detecting, staging, and monitoring sleep in people using wearable devices such as smartwatches. Systems and methods of the disclosure are device-agnostic in that different makes and models of devices may be used by the end-user patient/subjects, while the system functions consistently even if and when people use different devices. Systems and methods of the invention address situations in which people may use different wearable devices that record data in different ways, e.g., with different sampling frequencies, or different dimensions, or even devices that record different data. Systems and methods of the disclosure address different data types and formats as part of data collection when receiving patient data and preprocessing the data for presentation to analytical algorithms, or for comparison among data sets, or for storing data records for review and use by a clinician.

The invention provides systems and methods that stage sleep in patients using wearable devices regardless of the brand or features of the wearable device. Data collected from wearable devices are assigned a sleep stage regardless of manufacture or mode of operation. Methods provide precise and accurate sleep stage information to physicians via an online portal of the system, offering patients suffering from poor sleep opportunities for better medical outcomes. Methods include preprocessing data for a subject from a first sensor on a first wearable device; based on a type of the wearable device into standardized data and analyzing the standardized to identify sleep stages. Second data from a second wearable device with different formats or different content is also preprocessed a format matching that of the standardized data.

For example, a user may use a first wearable device such as the smart watch sold under the trademark APPLE WATCH SERIES 6 by Apple, Inc. (Cupertino, CA). The first device may record data from the user such as hear rate or motion, e.g., recording at a certain frequency. A user (the same or a different person) may use a second wearable device such as the smart watch sold under the trademark FITBIT SENSE by Fitbit, Inc. (San Francisco, CA). Systems and methods of the invention receive data recorded by either or both of the first device and the second device, as well as optionally any others, and analyze sleep patterns in those data.

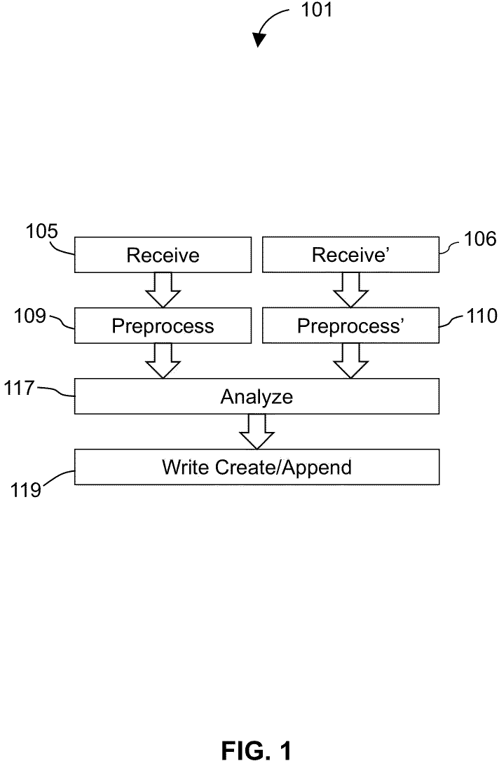

FIG. 1 diagrams steps of a method 101 for analyzing sleep patterns. The method includes receiving data for a subject from a first sensor on a first wearable device. A computer system receives 105 the data. The computer system preprocesses 109 the data based on a type of the wearable device into standardized data. For example, in some embodiments, the computer system operates (e.g., simultaneously) to receive 106 device from a second device. The second device and the first device may have different hardware, firmware, or software, and may operate according to different standards. In some embodiments, the first device records heart rate data at a first frequency and the second device record heart rate data at a second frequency. The system may optionally pre-process 110 data from the second device, although in some embodiments, the data is stored in a format as-obtained from one of the devices and data are only preprocessed 109 when received 105 from other device of another type. The system will analyze 117 the data obtained from the device(s) to determine a stage of sleep at one or more different times for a patient and perform a write operation 119 to create a record or append to a record so that the record includes sleep stage information for a corresponding subject.

In general terms, the system creates a clinical record, i.e., data for use by a physician in clinical treatment of a patient. The analysis 117 is discussed in far greater detail below and throughout, but generally includes obtaining data measured from the patient at various times and using a classifier to assign sleep stages to those times. The data is received 105 from a wearable device such as a smartwatch. The system operates with more than one different type or manufacture of device to receive 105, 106 the data and perform the analysis 117.

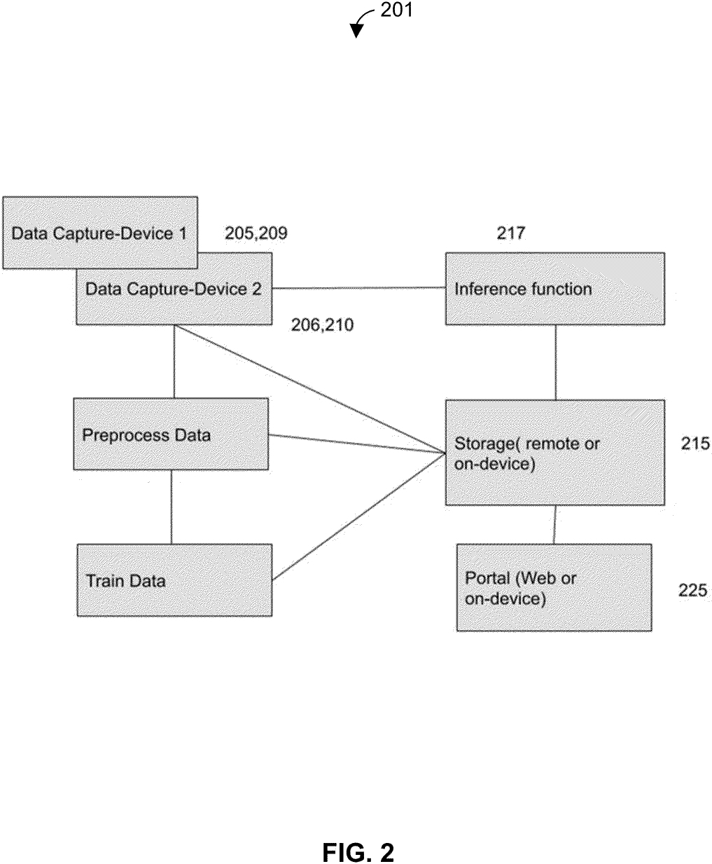

FIG. 2 is a diagram of a system 201 of the disclosure. The system 201 receives 105 data from a device 205. The device 205 preferably includes at least one sensor such as a photodiode. The device may also include one or more accelerometers. In some embodiments the device includes a photodiode and a light-emitting diode (LED) that operate as a photoplethysmography (PPG) sensor. Data is collected on the wearable device 205 and optionally transferred to a companion app on a personal computing device 209 such as a smartphone. The personal device 209 may be any suitable device such as the smartphone sold under the trademark IPHONE by Apple Inc., or that sold under the trademark GALAXY by Samsung. Data may be transferred from the wearable device 205 to the personal device 209 continually or in batches. Those data are preferably transferred from the personal device 209 to the system storage 215 continually or in batches. The system 201 also operates equally with a second wearable device 206, which itself collects data from a patient by making readings from sensors that may include PPG, accelerometers, others, or any combination thereof. The second wearable device 206 preferably captures the data as heart rate and activity and transfers the data to second personal device 210, which itself may be any suitable device such as the smartphone sold under the trademark IPHONE by Apple Inc., or that sold under the trademark GALAXY by Samsung. Additionally or alternatively, data may be alternatively sent directly from the wearable device to the storage system. Data can also be stored on a device for processing and not sent to the server.

Notably, the system 201 operates consistently with those devices 205, 206 and others being of any type or brand, even from different manufacturers or even device that output data with different file formats, different sampling frequencies, different units or scales, different transfer protocols, or different requirements for the presence of a personal device 210. For example, one feature of the disclosure is that data from the wearable device 205 need not flow (or “stream”) continually to the system storage 215. Instead, it may be that the wearable device 205 or the personal device 209 collects the data and uploads it to the system storage 215 periodically, or in batches. In some embodiments, the companion app on the personal device 209 initiates an upload periodically, e.g., once every 15 minutes, or every morning at a fixed time, or periodically and in response to a triggering event (e.g., the patient unlocks his or her phone). However, the second wearable device 206 and/or the second personal device 210 may transfer data to the system storage 215 according to a schedule that is different than for the first wearable device 205. For example, the second wearable device 206 may stream data directly to the system storage 215, while the first wearable device 205 transfers data to the personal device 209 every 15 minutes or every hour, and the personal device 209 transfers the data to the system 215 in certain intervals or when a useful network connection is detected. It is consistent with objectives of the invention that data from wearable devices 205, 206 need not be streamed continually to the system storage 215 at least because objectives of the invention include determining stages of sleep of patients using the wearable devices 205, 206, and presenting full records of that information to a clinician using a portal 225 of the system 201. While there may be optional modes by which the system processes the data continually, in certain preferred embodiments, it is only needed for data to be transferred to storage 215 periodically and the analyzed to generate 119 a report which is made available via the portal 225. Typically, data will be collected from the wearable devices 205, 206 during times that include nighttime, and the portal 225 will be accessed by a medical professional during business hours, e.g., during the data.

One feature of the system 201 is that it is operable to receive 105 data from a patient using the wearable device 205, and also to receive 106 second data from a second patient using a second wearable device 206. The system 201 is operable to identify sleep stages at different times for the patients even if the wearable devices 205, 206 output data with different formats or different content. For example, the wearable device 205 may sample heart rate (using a PPG sensor) at 1 Hz and the second wearable device 206 may sample heart rate at 0.2 Hz. The system 201 can receive one heart rate measurement for every second from the first wearable device 205, and one heart rate measurement every five seconds from the second wearable device 206. Other wearable devices may have other standards for fidelity or frequency. The system 201 may (e.g., via an inference module 217 associated with the storage 205 or directly by, e.g., a wrapper script or similar) preprocess the data. In some embodiments, the system preprocesses the data based on a type of the wearable device 206. E.g., where the first wearable device is a Fitbit sampling at 1 Hz, the system may recognize or be given information that the second wearable device 206 is an Apple watch sampling at 0.2 Hz. The system 201 may preprocess 110 data from the second wearable device 206 by operations that include interpolating heart rate data to provide data with 1 Hz (interpolated) heart rate data. By such operations, downstream analysis within the system 201 may proceed in a manner that is fully agnostic as to make or manufacture of the wearable devices 205, 206. The method 101 performed by the system 201 thus may include preprocessing 110 data based on a type of the wearable device 206 into standardized data and analyzing 117 the standardized data (e.g., with the computer system 201) to identify sleep stages at different times for a patient. It is notable here that the first wearable device 205 and the second wearable device 206 may be used by different people, i.e., different patients, at the same or different times, but also that the first wearable device 205 and the second wearable device 206 may be used by the same person. For example, a patient may change brands of device that they wish to use, and may stop using a first wearable device 205 when that person obtains and begins using the second wearable device 206.

Embodiments of the disclosure also work “app to cloud” and the system 201 may include or operate with a wearable device 207 that does not require a “smartphone app”. Any of the wearable devices 205, 206, 207, or others may communicate directly with system storage 215 without any personal device mediating the operations.

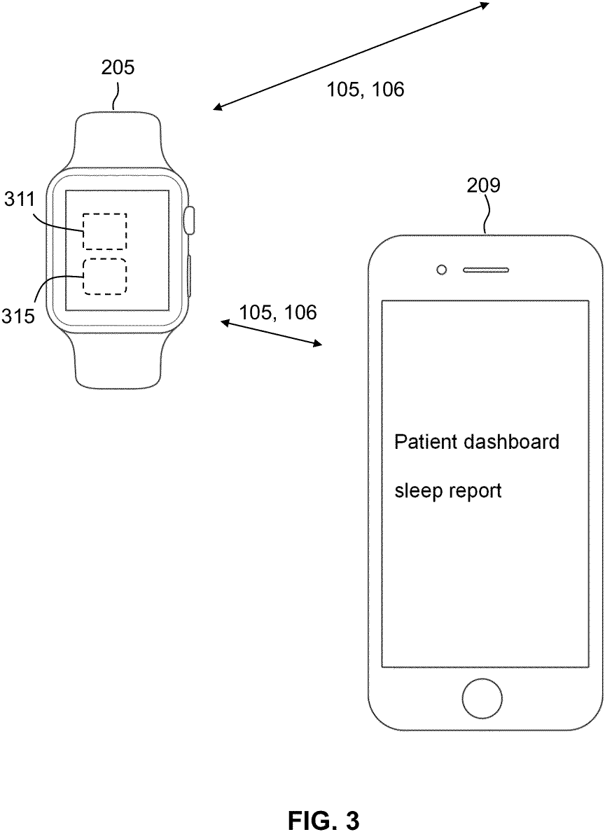

FIG. 3 shows a wearable device 205 and an optional personal device 209. The wearable device 205 preferably includes sensors such as either or both of a PPG sensor 311 and an accelerometer 315. Some embodiments of the disclosure may include one or more of either or both of the wearable device 205, 206 and the personal device 209, 210. That is, system and methods of the disclosure are operable with, and may include the first wearable device 205 and the second wearable device 206. Importantly, data may be received 105, 106 by the intermediating use of a personal device 209 or directly from the wearable device into the system 201. Either of both of the first wearable device 205 and the second wearable device 206 may include at least one photoplethysmographic (PPG) sensor 311 and at least one accelerometer 315. It may be that the first wearable device 205 and the second wearable device 206 output PPG and/or acceleration data with different formats, different content, or different sampling frequency. Any of those conditions may also be true of a third, or fourth, or yet other wearable device. A wearable device 205 may interact with the system 201 indirectly through a personal device 209, or the wearable device 205 may interact (e.g., exchange data with) the system 201 directly. Interactions and/or data exchange may use any combination of a local network (e.g., “Wi-Fi”), the Internet, and cellular data. As needed, the system 201 will preprocess second data from a second wearable device 206 into a format matching that of the standardized data. For example, the system 201 may capture heart rate (HR) data from a PPG sensor 311 on any device and, in all cases, regardless of the wearable device 205, preprocess the HR data to represent (e.g., by interpolation) 30 HR readings at 1 second intervals for 30-second epochs. Moreover, to support data capture from the wearable devices 205, 206, the system 201 may include one or more software modules that interact with each application programming interface (API) associated with different wearable devices from different manufacturers. The API may be part of a smartphone app in a software app on the personal device 209, and/or the API may be on the wearable device 205 directly. In any event, the system 201 sends instructions in data requests in a manner recognized by the API, based on a manufacturer, brand, or standard of the wearable device 205 or associated smartphone app. Regardless of the different formats, content, or sampling frequencies of the devices 205, 206, the computer system 201 is operable to receive data transfers from different first and second wearable devices 205, 206 used by respective first and second patients.

The system 201 operates to continue analyzing 117 standardized data to identify sleep stages at different times for the subject. In fact, data obtained from the first device 205 may be used in the same analysis as second data obtained from the second device 206. It does not matter that the data and the second data are output from the respective devices with different formats or different content (e.g., that the patient replaced a first device that samples heart rate at 1 Hz with a second device that samples heart rate at 0.2 Hz). Preprocessing 109, 110 those data standardizes the data in manner that is useful during the analysis 117.

Preprocessing may include a variety of steps, format changes, analytical components, or software operations. For example, as given above, pre-processing may include interpolating or standardizing readings from at least once device to a standard format. E.g., data from devices of various types can be preprocessed to match data from one device, or data from all device types may be preprocessed into a system-wide standardized format. Preprocessing may include operations that are applied to the data without regard to the values within the data (e.g., interpolating a sampling frequency) and/or may include operations that are specific to values within the data (e.g., internal normalization). Preprocessing may also standardize data despite non-uniform frequencies or patterns of transfer from device 205, 206 to system storage 215. For example, one wearable device 206 may transfer data to the system 215 every morning when a user first logs in to a personal device 210, whereas another wearable device 205 may save data as batches every 15 minutes and transfer those batches of data to the system storage 215 when a reliable internet connection is available.

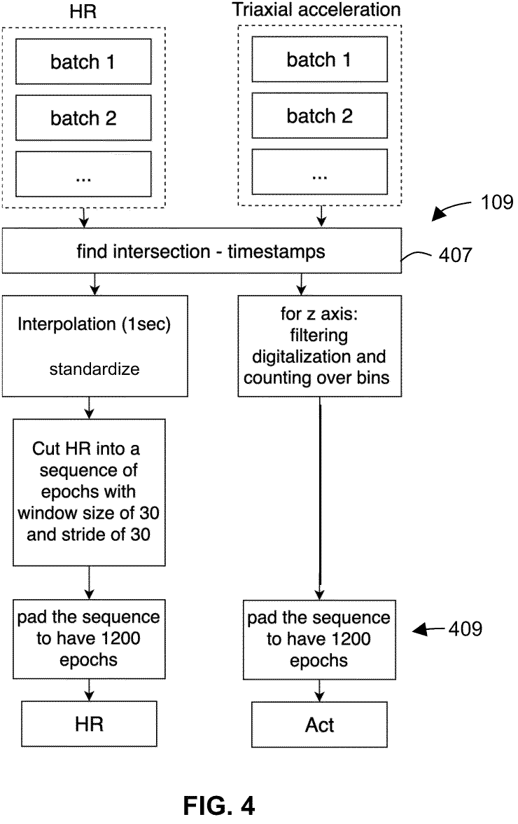

FIG. 4 diagrams preprocessing according to certain embodiments. As shown, a wearable device 205 makes heart rate (HR) measurements using a PPG sensor 311 and makes acceleration measurements using triaxial accelerometers 315. Those data are collected in batches (e.g., every 15 minutes) and stored on the wearable device 205 and/or the personal device 209. When a connection is available, batches or HR and triaxial acceleration data are transferred to the system storage 215. Preprocessing 109 may include operations such as finding intersections 407 among the batches based on timestamps and/or concatenating the batches into a uniform ordered chronology of HR and triaxial acceleration measurements from the patient over time. The HR data may be interpolated (e.g., to represent one heart rate measurement per second) and optionally standardized according to other suitable operations (e.g., for example, a patient-specific correction may be applied or quality control metrics or filters may be applied to, e.g., remove outliers or data that is flagged for removal by a quality score operation). The numbers shown in the figure are exemplary and systems and methods of the disclosure may work equally well with varying epoch lengths, strides, interpolations, etc. For example, epochs may have window size of 30 s, or of 50 s, or of 10, or of 40, or any other suitable value. Stride may be 30, 50, 10, 50, or any other suitable value. Interpolation may be to any suitable frequency.

In certain embodiments, the incoming data are assigned to epochs, which may predefined spans of time useful to downstream analyses 117. For example, where heart rate (HR) is measured at or interpolated to 1 Hz, the incoming time span of data may be assigned to windows or bins. Window with a size and stride and bin are terms understood in the literature for data analysis including classification and machine learning, and may be used as used in Dehghani, 2019, A quantitative comparison of overlapping and non-overlapping sliding windows for human activity recognition using inertial sensors, Sensors (Basel) 19(22):5026, incorporated by reference. Generally, window or bin applies to a subset of a set of data with the implication that, for linear (e.g., over time) data in particular, values of those data will be put into sets, dubbed windows or bins (overlapping or not), where those sets are suitable to an analysis, e.g., as inputs to a classifier or other such analytical algorithm. In some embodiments, the system 201 cuts (e.g., divides the contents of the digital data) the heart rate data into a sequence of epochs corresponding to sliding windows over the heart rate data from over time. The window may have any suitable size and stride such that each epoch includes one sequence of the data. E.g., where the window size is 30 and the device 205 measures heart rate at 1 Hz (or is interpolated to once Hz), each epoch may span 30 seconds and include 30 heart rate measurement values. Accelerometer data may be treated similarly, e.g., optionally filtered and/or digitized and divided into bins. In certain embodiments, the accelerometer data is digitized per epoch so that each epoch will have one single “activity” value. Thus, for example, it may be that the data are divided into epochs (e.g., 30 s) with 1 Hz heart rate (e.g., 30 values) and some arbitrary number (e.g., 1) of activity value(s) for that epoch. The data may be held (in system storage 215) as sequences of epochs.

The preprocessing 109 preferably includes padding 409 the heart rate (HR) and activity (Act) data. Padding makes all sequences in a batch fit a given standard length (e.g., 1200 epochs). In the illustrate example, there is one heart rate (HR) value per second, the window size is 30 so each epoch represents 30 seconds of measurements made on a patient wearing the wearable device 205. Each epoch includes 30 HR values and 1 activity value. Those values represent the person’s heart rate over that 30 s and a measure of how much body or limb motion the person exhibited in that 30 seconds. As shown, each sequence may be 1200 epochs, or 1 hour. Other values for epochs or sequences may be used. In the depicted embodiment, each sequence is padded and represents one hour’s worth of measurements from the person with significant potential information about that person’s sleep state(s). The preprocessing 109 provides padded sequences of HR and Act data.

Systems and methods of the disclosure include features and functionality that add clinical benefits to the ability to capture batches of data recorded from wearable devices. One clinical benefit of systems and methods of the disclosure is that they are interoperable with disparate physical wearable devices, the operating systems of those devices, or the data collection standards or formats provided by those devices. Systems and methods of the disclosure output clinical data via a portal 225 useable by a clinician to identify or diagnose sleep-related conditions, guide therapeutic choice for sleep related conditions, monitor therapeutic efficacy, and counsel patients throughout treatment. To that end, a clinician (e.g., user of portal 225) may have no preference regarding, or interest in, what type or brand of wearable device a patient is using. To give the clinician relevant reports and data, the system includes modules and functionality that make the back-end of the system 201 device agnostic because the system is operable with different device and may preprocess 109, 110 data into a regular format that can be analyzed 117.

Another clinical benefit of systems and methods of the disclosure is the option to use circadian models for a patient by which data from day, or wake time, and night, or periods of sleep, are both used to both improve sleep staging and also to detect sleep-relevant activity during putative day, or wake time. The use of a circadian model may be implemented in different ways with different particular details, but will in general use data obtained for a patient via a wearable device over time that includes both day and night.

Circadian rhythms are biophysiological phenomena in plants and animals. Genetic components have been found to operate in many cell types in mammals with effects apparently largely coordinated by pacemaker neurons. The biological rhythms are primarily influenced by environmental light-dark cycles. As used herein, a circadian cycle may refer to one cycle of light and dark, e.g., one day, one period of the cycle. For purposes of systems and methods of the disclosure, data representing a circadian cycle need not be obtained for a full 24 hours. In general, it may be enough that a substantial number of hours of light, or “wake”, time are obtained and similarly for dark, or night, time. A circadian rhythm is endogenous to a organisms, e.g., the patient. As used herein, a circadian cycle is—strictly speaking—a diurnal rhythm, is it refers to an extrinsic periodicity to which it is hoped that a patient entrains. That is, in some embodiments, methods of the disclosure collect data according to a model of a circadian cycle (a natural diurnal cycle that need not be 24 hours, so long as a substantial amount of putative light and dark times are sampled). Preferably a substantial portion of each cycle (e.g., at least 20 hours) is sampled. The data from the patient is used to build a circadian model of the patient. As used herein, circadian model may be understood to include a digital computer-based model of biophysiological information from a patient that is captured over a plurality of circadian cycles (e.g., light-dark periods), preprocessed, and stored in a such a way as it is useful to analyze or represents the patient’s circadian rhythms, or aberrations thereof. The circadian cycle is a useful term to describe the sampling period, and could be manifest in, or found in, test data, or made-up data. A circadian rhythm is an endogenous biological phenomenon of a patient (or in other organisms).

In certain embodiments, a circadian model is built for a patient by collecting data over a plurality of circadian cycles. The circadian model may change over time and, in fact, would be expected to change if the system 201 and method 101 are being used, for example, to monitor efficacy of a sleep therapeutic. The circadian model is personalized to a patient and is preferably stored in that patient’s personal profile along with other data of the disclosure. Analysis of the circadian model may potentially reveal correspondence between the patient’s circadian rhythm and extrinsic environmental light-dark cycles. The circadian model provides a measure of the patient’s physiology in light and dark periods and is useful, once built, to classify episodes of patient sleep with accuracy and precision. An insight of the disclosure is that by building the circadian model to include daytime measurements as well as nighttime measurements, and in a personalized manner, then when the model is used to classify episodes, the classification is more accurate and precise for sleep/dark periods than if performed without a model that was built using wake/light periods.

For ease of reference and reading, this disclosure may refer to behavior of a human as sleep or wake and as occurring during night or day, or during dark or light. To make reading easy, some examples may equate nighttime with sleeping and daytime with normally being awake or intending to be awake. It is recognized that different people keep different schedules and the principles herein are equally applicable. For example, for a patient who works a graveyard shift from 11 pm to 7 am and normally attempts to sleep from 8 am to 4 pm, if an illustrative example in this disclosure uses words referring to sleeping at night, detecting daytime sleep, for that person, the example may be taken to refer to sleeping between 8 am and 4 pm and detecting sleep between 4 pm and 7 am.

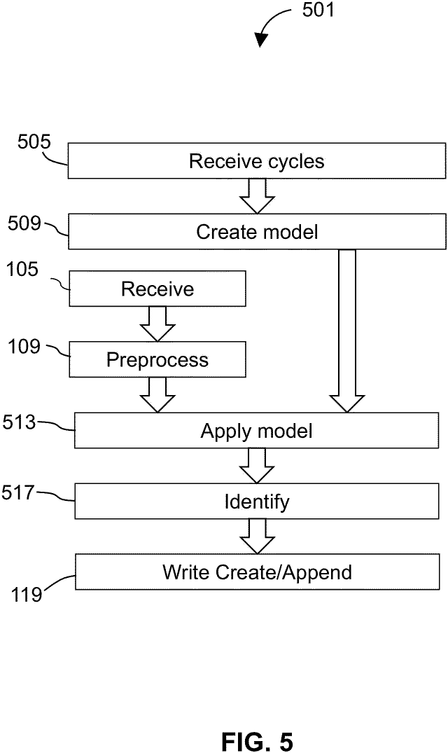

FIG. 5 diagrams steps of a method 501 of analyzing sleep using a personalized circadian model. The method 501 includes receiving 505, from a wearable device used by a patient, heart rate data from over a plurality of circadian cycles. A computer system 201 may calculate a distribution of values in the heart rate data from multiple hours of wake time and sleep time in each of the plurality of circadian cycles and create 509 a circadian model for the patient. The circadian model preferably includes a resting heart rate value for the patient and a defined operation for applying sleep labels to new data from the wearable device. The circadian model preferably also includes a threshold value for a heart rate variability and optionally also a threshold value for other measures such as activity, dissolved oxygen, sound (e.g., dB), light levels, others, combinations thereof. The circadian model is preferably saved in a personal profile for the patient, within system storage 215. That personal profile thus includes data measured from the person for a plurality of day hours and a plurality of night hours. Moreover, the received 505 data includes a plurality of cycles of that data, so that the circadian model can reveal information about the circadian rhythm of the patient. That circadian model is then used in ongoing analysis to detect and stage sleep in the person. The method 501 includes receiving 505 test data from the wearable device and applying 513 the circadian model to the test data to identify 517 sleep intervals. Here, “test data” is used to distinguish from the ongoing sleep data that is analyzed to detect and state sleep, as compared to the rolling, multi-day period used to build the circadian model.

Once the circadian model is built, the patient may use his or her wearable device 205, 206 to detect and stage sleep. The system 201 may use incoming data for both model building and ongoing sleep analysis. The circadian model may be built of some number of prior cycles of data, but one sleep interval may also (e.g., independently) be being analyzed for sleep staging. In such cases, the operation of one component of the system 201 (model building or sleep staging) need not have any immediate influence on the other). An important feature of the circadian model is that is uses data recorded from the person for a number of hours from a number of days in building the model used to stage sleep. That model may be implemented in different ways, or different variables or numbers may be adjusted.

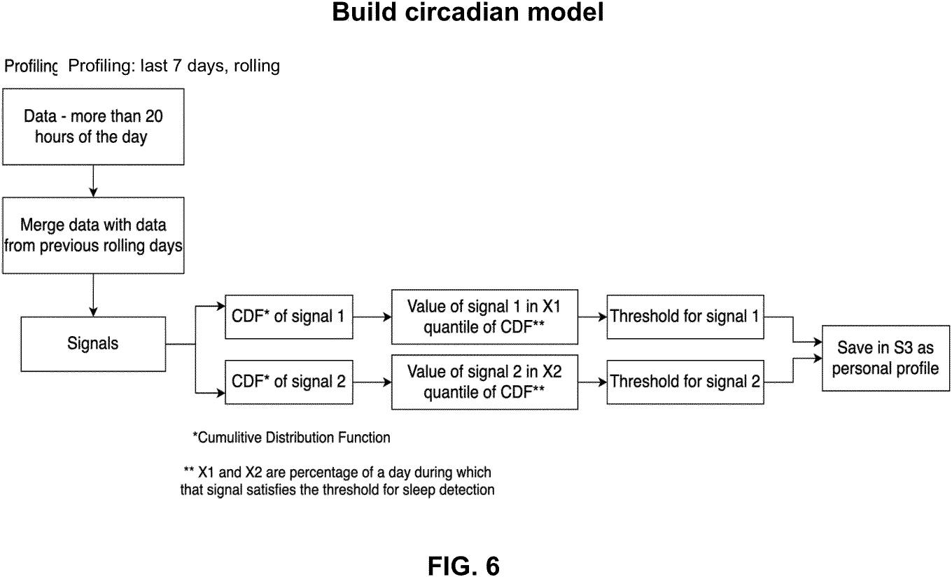

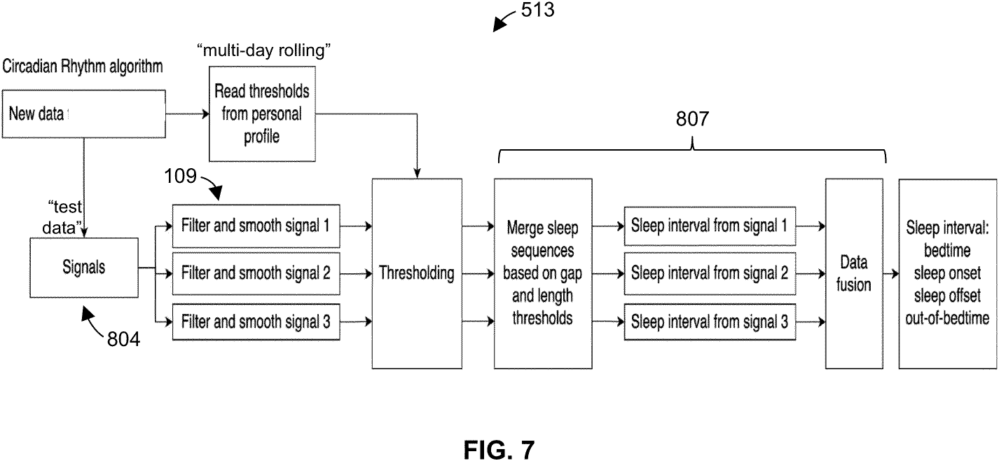

FIG. 6 shows how a circadian model is built according to certain embodiments. In the particular, depicted embodiment, a wearable device is used to profile the patient over a rolling span of the prior seven days. To benefit from the diurnal nature of a circadian period it may be preferable that data from each circadian cycle includes a substantial number of hours from day and a substantial number of hours of night. As shown in the diagram, the model may use, e.g., at least 20 hours from each day. That measure of at least about 20 hours of each day has been found to work and is consistent with current models of wearable devices such as smartwatches and the amount of time they may use for battery charging. As new data come into the system 201, the system merges data from each new day with the prior rolling span of N-1 days, for some rolling period of N days (e.g., 7). For the N days in the rolling the period, the system 201 extracts signals, .e.g., one signal, two signals, three, or more, such as for heart rate and/or activity measures. An important feature of the circadian model is the inclusion of daytime data allows thresholds to be set for certain metrics, where the thresholds are good for distinguishing among stages of sleep. In the depicted embodiment, the circadian model for the patient will include an HRV threshold and a threshold or function for activity, used in examining later “test data” for meeting or exceeding such thresholds. As shown, the depicted approach analyzes a cumulative distribution function of the signals. References such as Xn may be used, where Xn is or includes a percentage of a day during which a signal satisfies the threshold for sleep detection is determined. The system determines a value of the one or more signals in an Xn quantile of the CDF to establish a threshold for each signal under analysis. For example, a value such as the identified/determined quantile of the cumulative distribution function of heart rate can be saved in the circadian model as a resting heart rate. Those values, as well as optionally a threshold value for activity are saved in the circadian model. The circadian model may be saved as part of a the personal profile of a patient in the system storage 215. Notably, in certain embodiments, the model is not static but changes over time. E.g., each day, when the prior 7-day rolling period is updated, the model is updated. This is useful because the patient may be undergoing therapy and that patient’s circadian rhythms may be changing, meaning that his or her circadian model may change, and the system 201 records and documents and uses that change over time to give clinical grade data to the clinician via the portal. The patient-specific circadian model is saved in the system storage 215 and used in sleep staging. The system 201 benefits from using the circadian model because the later analysis of patient sleep is grounded in physiological data from that patient’s day and night cycles of data. FIG. 7 is a workflow showing how the method 101 for analyzing sleep patterns may use the circadian model to identify sleep stages at different times for the subject. The workflow illustrates the method 501 of analyzing sleep using a personalized circadian model. Specifically, the method 501 includes receiving 505 test data from the wearable device and applying 513 the circadian model to the test data to identify a sleep interval. Across the top fork of the workflow is represented the model building. The multi-day rolling set of date are obtained (e.g., preferably daily, but once at a minimum, optionally every week or so). The computer system 201 creates 509 a circadian model for the patient, preferably including at least a resting heart rate threshold, and has the model saved in the patient profile in system storage 225.

Test data (e.g., some night later, once the profile is in place) comes to the system 201 as signals. As discussed above, it is not imperative that the data stream in in real time, continually from a smartwatch or other wearable device 205. Those data may be passed as batches, e.g., from wearable device 205, optionally to a personal device 209, and on into the system 201. The test data may include signals such as heart rate and accelerometer obtained by the wearable device 205 as values 804. The system preferably preprocesses 109 the incoming data. Preprocessing is one step that may aid the system in working with multiple, disparate devices 205, 206. The pre-processing may include interpolation, standardization, quality filtering, smoothing, or other such operations, in any combination. Thresholds from the circadian model are applied 413 to the test data to identify sleep sequences. For example, each of heart rate, HRV, and activity (or a function of any of those such as f(activity)) may be subject to a comparison to a threshold. In some embodiments, sequences of data (e.g., sequences of epochs) that meet thresholds are identified as sleep sequences. Through a series of merger and fusion operations 807, a sleep interval for the patient is identified. Any or all of the merger and fusion operations 807 may be performed by an inference module 217 of the system 201. A significant detail is that the input to the operations 807 includes threshold comparisons for measurements from PPG sensors and accelerometers from a plurality of epochs (e.g., batch 1, batch 2, etc.). The operations concatenate and merge sequences for heart rate and activity and fuse those (e.g., by a majority-wins rule per epoch) to output a chronology that includes a sleep interval. Here, sleep interval is held in system storage 215 as, e.g., a comma-separated value file and preferably includes records of at least four events per night: go-to-bed time, go-to-sleep time, wake-time, and rise-time. An important clinical benefit of systems and methods of the disclosure is embodied in the treatment of sleep interval as included those four distinct times. It is recognized and intended that people may be helped best by providing clinicians with records that include, for example, nightly records of when the person went to bed and when he or she actually went to sleep.

Systems and methods of the disclosure analyze 117 data received 105 from a wearable device 205 to identify sleep stages at different times for the subject. This identification process is sometimes referred to as sleep staging. Accurate identification of sleep stages is valuable in the diagnosis and treatment of sleep disorders as well as for monitoring therapeutic efficacy. Systems and methods of the disclosure may be used to classify sleep into one of three, four, or more stages. For example, three-stage staging can classify epochs as one of wake, non-REM sleep (NREM), or REM sleep. Additional stages can further classify NREM according to depth of sleep. Clinically useful assessment of sleep benefits in particular by reliably identifying wake periods after sleep onset. Prior art approaches to sleep staging require the use and analysis of electro-encephalogram (EEG) and/or electrooculogram (EOG) recordings. While such tools may be beneficially included and used in systems and methods of the invention, they are not required, and systems and methods of the invention operate to identify sleep stages in data collected by wearable devices including, e.g., from PPG sensors and accelerometers. Importantly, systems and methods of the invention are built to be interoperable with different devices (e.g., different brands, from different manufacturers, that capture data in different formats or with different sampling frequencies) and preferably also to use personalized circadian models to improve the identification of sleep sequences. The system performs operations 807 that give a patient’s sleep interval, including between, sleep onset, sleep offset, out-of-bedtime, and epochs within that interval.

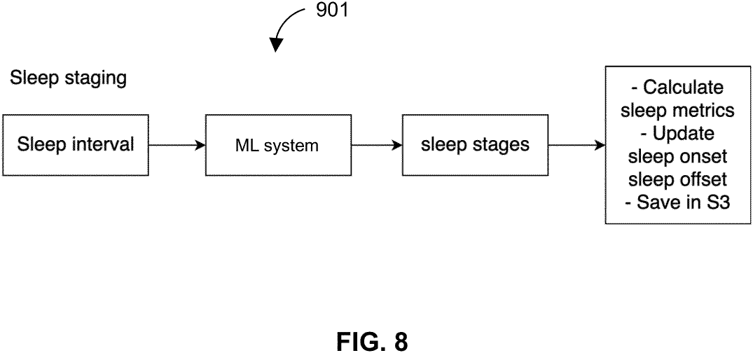

FIG. 8 shows the steps in sleep staging according to the methods 101, 501. The sleep intervals are provided to a machine learning system 901 that assigns a sleep stage to each of a plurality of epochs in the sleep interval (e.g., standardized data for that patient). For each epoch, the machine learning system 901 assigns a sleep stage. For example, in three-stage embodiments, the system 201 classifies each epoch as one of wake, REM sleep, and non-REM sleep (NREM). Preferably, the inference module 217 assigns sleep stages and probabilities, and may also record certain metrics of sleep quality (e.g., duration of sleep, duration of REM, whether certain patient-specific goals are met). The inference module 217 may also, under guidance form the machine learning system, update the sleep onset and sleep offset first written in the sleep interval. The output of the machine learning system 901 includes accurate and precise high-quality sleep stage labels applied over epochs throughout the night for a patient and saved within the profile with the sleep interval with correct go-to-bed time, sleep onset, wake-up, and rise-time. The data received 105, 106 by wearable device 205, 206 was analyzed using patient-specific circadian models that include sensor data from a substantial number of hours from each of a number of days for each patient. Including the daytime hours in the circadian model improves the ability of the system 201 to detect sleep relevant episodes, or sleep sequences, at any time. To give but one simple but illustrative example, for a patient who reports to her doctor that she has difficulty sleeping at night, and reports having no significant daytime naps, the system 201 using the circadian model could potentially inform the doctor of either or both of facts that the patient is, in fact, taking long daytime naps with long periods of REM sleep or is sleeping for much longer at nighttime than is self-reported (reliability of detection of nighttime sleep much improved by using the thresholds built into the circadian model using both night and day data). The accurate and precise high-quality sleep stage labels applied over epochs throughout the night for a patient along with the sleep interval with correct go-to-bed time, sleep onset, wake-up, and rise-time are output from the sleep staging operation and written to the system storage 215.

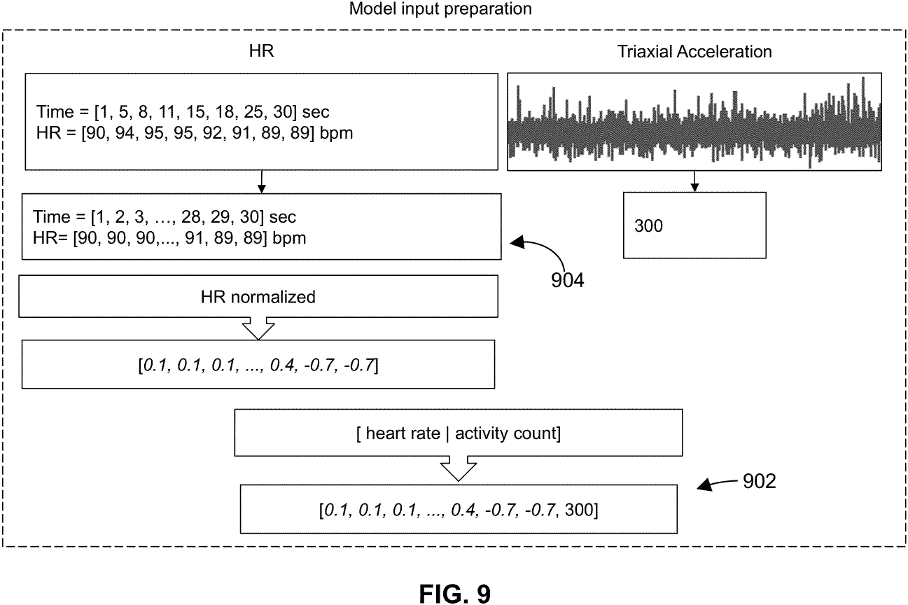

FIG. 9 shows preparation, or preprocessing, of input to a machine learning model according to certain embodiments. Other embodiments and details are within the scope of the invention. In the depicted embodiment, measurements of heart rate (HR) and triaxial acceleration are received, e.g., from sensors on a wearable device 205, 206. It may be preferable to initially process 19 the HR data values 804 into a uniform sampling frequency with interpolated values 904 (e.g., in bpm). The data are optional standardized or normalized in some manner. In certain embodiments, triaxial acceleration data is also received, e.g., as a numerical value for activity count. The HR and activity count may be concatenated together and extracted as features 902. In the depicted embodiment, each feature 902 includes 30 interpolated HR values 904, one per second, and an activity count, with the feature representing one epoch. The features 902 are suitable as inputs to a machine learning system 901. Any suitable tools or structures may be used for the machine learning system 901. For example, the machine learning system 901 may include a neural network, a random forest, a support vector machine, other sleep stage classifier, or other suitable machine-implemented algorithm. The machine learning system is preferably trained on training data. For background, see Sun, 2017, Large-scale automated sleep staging, Sleep 40(10):zsx139 and Korkalainen, 2020, Deep learning enables sleep staging with photoplethysmogram for patients with suspected sleep apnea, Sleep 43(11):zsaa098, both incorporated by reference. It is noted that systems and methods of the invention may be device-agnostic at least in that feature 902 has the same structure for different manufacturers of devices even when the inputs to the depicted preprocessing have structures unique to the various devices. For example, different devices will give HR measurements at different times (i.e., other than at 1, 5, 8, 11,... seconds as shown) and may give acceleration in different axial system than, e.g., triaxial (e.g., along one axis, or two, or six...). In the depicted preprocessing, the features 902 may have a standardized size, dimensionality, arrangement, or content.

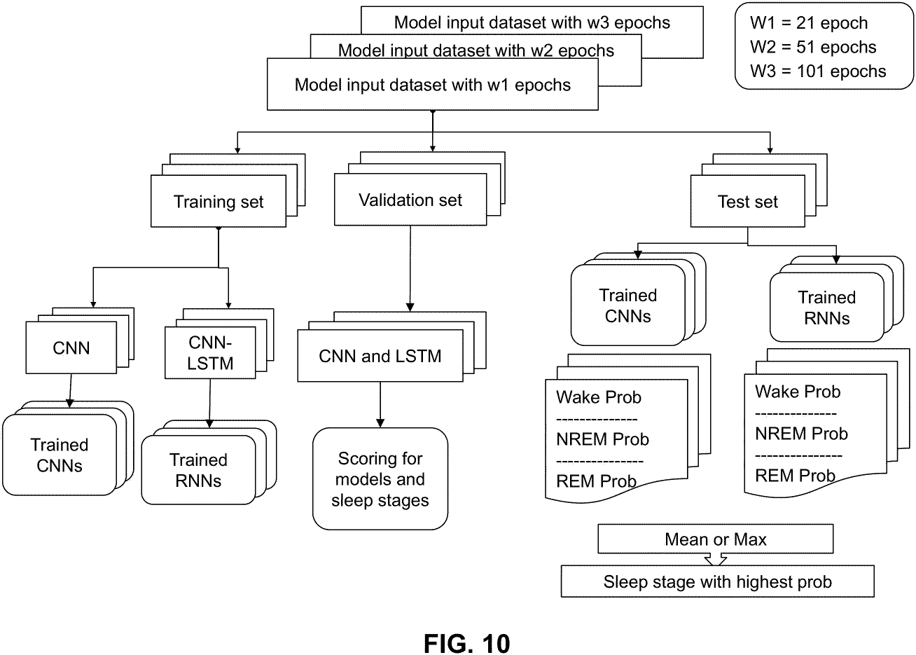

FIG. 10 shows use of a machine learning system 901 of the disclosure in training, with training data, in validation, with validation data, and as applied to patient test data. Preferably, the machine learning system 901 receives input datasets in sets of epochs. The tree presented shows different important functions that may involve the machine learning system 901. The machine learning system 901 may be given a training set. Any suitable training data set may be used. For example, the machine learning system 901 may be trained using a data such as that from the Multi-Ethnic Study of Atherosclerosis (MESA). MESA is multi-center longitudinal investigation of factors associated with the development of subclinical cardiovascular disease with participants also enrolled in a Sleep Exam (MESA Sleep) which included full overnight unattended polysomnography (PSG), 7-day wrist-worn actigraphy, and a sleep questionnaire. The objectives of the sleep study are to understand how variations in sleep and sleep disorders vary across gender and ethnic groups and relate to measures of subclinical atherosclerosis. For sleep-staging purposes, PSG data may be used as a gold-standard data such that the MESA Sleep data provides a labeled training data set. The training data is preferably used to train the machine learning system 901.

Whatever architecture is implemented for the machine learning system 901, once the system is trained, it may be preferable to perform validation. Validation may include exposing the machine learning system 901 to a set of validation data, e.g., gathered from a participants in a validation study. The validation participants preferably use the system 201 for its features, including its ability to work with data from wearable devices 205, 206 from different manufacturers or with different operational specifications. The validation set of data preferably also includes circadian models built in a patient-specific manner. If it is intended, then when the machine learning system 901 operating in the validation stage meets objective standards (e.g., concordant with PSG data from validation study participants), then the machine learning system 901 may be used in an ongoing basis on test sets that include test data. As shown, the machine learning system preferably includes one or more of some form of convolutional neural network (CNN) and recurrent neural network (RNN). The machine learning system 901 operates on the test data to classify epochs from the patient into stages of sleep. In an embodiment, the system 901 uses three-stage classification, identifying each stage as wake, NREM, or REM. In certain embodiments, the system 901 uses HR, HRV, and Act. Optionally, those inputs are classified independently, and the machine learning system 901 takes the mean or maximum congruent independent classifications to assign the sleep stage with the highest probability.

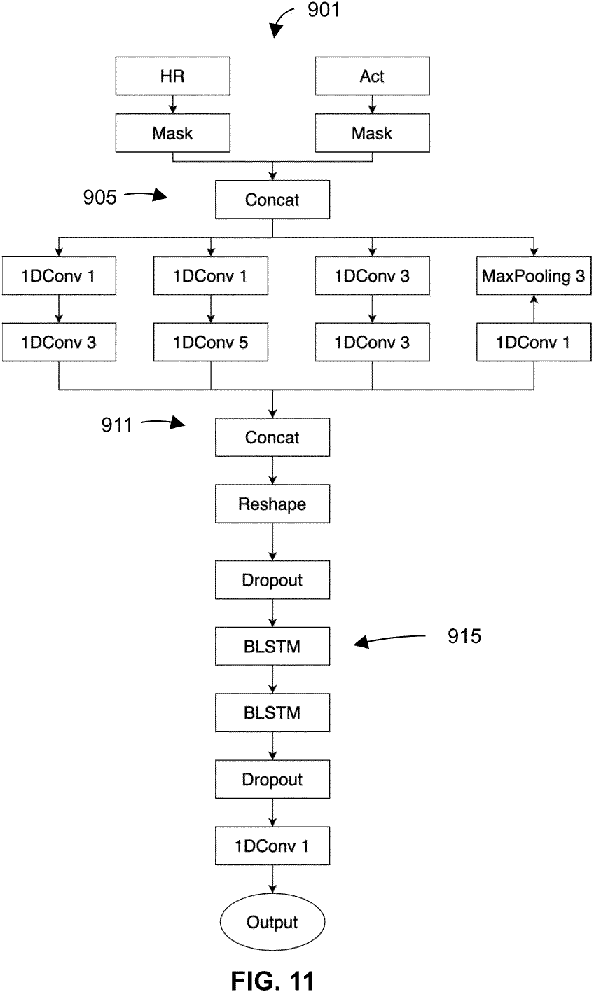

FIG. 11 gives one exemplary architecture that was built for the machine learning system 901. As shown, masking may be applied to the padded HR and Act data, to inform the machine learning system 901 that some part(s) of the data is padding and need not inform the analysis. A module of the machine learning system 901 concatenates 905 the masked and padded HR and Act data. Those data may be passed to a suitable machine learning algorithm. Any suitable algorithm may be used. As depicted, the system 901 uses 1D convolutional neural networks (1DConv). Pooling (here shown as max pooling) is employed after the 1DConv layers to reduce dimensions. Output vectors are concatenated 911 and optionally reshaped. Reshape may be provided as a function available within a machine learning system 901. See Gulli, 2019, Deep learning with TensorFlow 2 and Keras: Regression, ConvNets, GANs, RNNs, NLP, and more with TensorFlow 2 and the Keras API, 2nd Edition, Packt Publishing, Birmingham, UK 612 pages, incorporated by reference. The machine learning system 901 may further include a dropout layer, which aids in preventing overfitting. Dropout assigns a zero weight to one or more neurons and literature shows that dropout layers remove noise and improve results.

Importantly, the machine learning system 901 may include one or more operations or layers that are informed by time-dependencies within the data. The machine learning system 901 includes modules, layers, and an architecture that captures and is informed by time-dependencies in the sequential data (e.g., HR and Act). The machine learning system 901 may include one or more time-dependency layer 915 or algorithms specifically designed to capture time-dependencies such as, for example, a long short-term memory (LSTM), a bi-directional LSTM (BLSTM), a recurrent neural network (RNN), temporal convolutional network (TCN), others, or combinations thereof. A BLSTM is an example of an LSTM. By comparison, in convention feed-forward networks, when classifying a particular interval, there is no consideration by the model of prior (or subsequent) intervals. A dependency on past (and future) intervals can be introduced by creating recurrence within a neural network, where output of a layer is fed into that layer. Capturing those time dependencies across HR and Act data is valuable for sleep staging. In the depicted embodiment, the machine learning system 901 includes two BLSTM layers, followed by a dropout and a 1DConv. The output from the system is a sleep stage classification. The classification may be performed using three classes: wake, NREM sleep, and REM sleep. However, other sets of classes (e.g., wake, NREM light, NREM deep, and REM) may be used. The machine learning system 901 may be implemented using any suitable environment and tools such as, for example, Python 3.6 using Keras API 2.24 with TensorFlow 1.13.1backend.

Thus, using the machine learning system 901, the system 201 identifies what sleep stage the patient is in at recorded times (e.g., epochs), while the patient uses the wearable device 205. As described, the machine learning system 901 preferably includes an ensemble of classifiers that capture time-dependencies and convolutional neural networks, such as, e.g., BLSTM and 1DConv. The input to the machine learning system 901 preferably includes features 902 and the outputs are preferably sleep stage classifications over time for the patient uploaded into a clinical data system available to a clinician through a portal 225 of the system 201.

Sleep analysis using the personalized circadian model preferably includes profiling to create 509 a circadian model for the patient, sleep detection by receiving 505 test data from the wearable device and applying 513 the circadian model to the test data to identify sleep periods, and multi-staging by which a machine learning system 901 classifies epochs into sleep stages for the patient. At profiling to create 509 a circadian model for the patient, data such as HR and/or activity are received 505 from a wearable device 205 used by a patient from over a plurality of circadian cycles. A computer system 201 may calculate a distribution of values in the heart rate data from multiple hours of wake time and sleep time in each of the plurality of circadian cycles and create 509 a circadian model for the patient. The circadian model preferably includes a resting heart rate value for the patient and a defined operation for applying sleep labels to new data from the wearable device. The circadian model preferably also includes a threshold value for a heart rate and a function for identifying sleep from activity measurements. The circadian model is built from that data measured from the person for a plurality of day hours and a plurality of night hours. Moreover, the received 505 data includes a plurality of cycles of that data, so that the circadian model can reveal information about the circadian rhythm of the patient. Once the circadian model is built, the patient may use his or her wearable device 205, 206 to detect and stage sleep. For sleep detection, test data are received 505 from the device 205, and the circadian model is applied 513 to identify 517 sleep periods, or intervals. Preferably, the output of the sleep detection is at least one sleep interval including four characteristic time points: go to bed, go to sleep, wake, and rise. At multi-staging, the machine learning system 901 classifies epochs of the sleep interval into sleep stages for the patient. Using the machine learning system 901, the system 201 gives a result that includes sleep stages for the patient through nights of sleep. When multi-staging is 3-stages, the assigned stages may be wake, NREM, and REM. The result may include other outputs than the assigned stages. For example, the result may include probabilities assigned to the stages. The result may optionally include metrics of sleep, including e.g., total duration or whether certain objectives of the patient and clinician are being met. The result may include summaries or identifications of problematic outliers from the heart rate and/or activity data. The result may include multiple nights' and days' worth of information, organized or saved according to, e.g., a preference of the clinician. Systems and methods of the invention are useful for capturing and evaluating clinically-actionable sleep information from patients. In general, a patient may use a wearable device 205, optionally under control of a related mobile app on personal device 209. That same patient at another time, or another patient, may use another wearable device 206. The implementation of the system 201 is agnostic as to manufacture or function of the wearable devices 205, 206. The system 201 may preferable provide a portal 225 by which a clinical such as a physician may access and use the clinical information about the patient provided by the system 201.

The system 201 includes functionality written to interact with the devices 205, 206 and other devices according to the application programming interfaces (APIs) of those wearable devices or their associated personal device 209, 210 apps.

The described systems and methods may include a number of features and functionalities that may be implemented in any of the embodiments. For example, the system 201 may include the modules or shell scripts written to interact specifically with each API associated with different wearable devices 205, 206. For example, it is common that wearable device will have an app on the wearable device 205 and a related “companion app” on a personal device 209. Typically, the manufacturer will publish or make available API standards that are associated with that device to allow developers to build functions that use sensors on the devices. Here, the system 201 may include modules or scripts for interacting with the APPLEWATCH and the FITBIT, and may further include other API functionality.

Using such functionality, the system 201 may issue instructions that control how the wearable device 206 operates. For example, it may be found that it is preferable to the system 201 to sample heart rate at 1 Hz. Moreover, there may one model of wearable device 205 that, by default, samples heart rate at 1 Hz, but there may be another model of wearable device 206 that samples heart rate by default at some other frequency, e.g., 0.2 Hz. However, the manufacturer may provide the second wearable device 206 with an optional, non-default mode of operation that changes the HR sampling frequency. For example, some wearable devices come with a “workout mode” for use by athletes who prefer to see heart rate information at a high frequency. In some cases, it may be found that in “workout mode”, the wearable device samples heart rate at a higher frequency than the default 0.2 Hz, e.g., at 1 Hz or even higher. The system 201 may include a software module or script that instructs the wearable device 206 to operate in the optional mode (e.g., “workout mode”) to collect data best-suited for systems and methods of the invention.

Other features and functionalities may be implemented in any of the embodiments herein. For example, by including the circadian model building in the system 201, systems and methods of the invention are naturally suited for detecting sleep during the daytime. The system 201 may use the circadian model and the classifier to detect episodes of daytime sleep by the patient. Systems of the invention built with a machine learning system 901 are tested for performance. In the tested system, the machine learning system includes a classifier comprising an ensemble of LSTM and 1DConv layers. The extracted features include an activity count and interpolated 30 sec of HR at 1 Hz. Three-stage classification is performed with wake, REM, and NREM, with windows of 21, 51, and 101 epochs. The MESA Sleep data is used to train the machine learning system 901 over 1,297 training subject, and then tested on 324 subjects. The MESA Sleep data is labeled training data from multiple subjects. The tested system according to methods and systems of the disclosure obtained 79.78 accuracy, 88.58 specificity, and 72.18 sensitivity. It may be found that those accuracy, specificity, and sensitivity values are provided by using an ensemble of classifiers that include one or more layers that capture time dependencies, such as recurrent neural networks, LSTM or BLSTM layers. The machine learning system 901 outputs records of sleep stages or intervals for each subject, wherein contents of the records are consistent regardless of the different formats or different content in data from different wearable devices 205, 206. Implementing features or details or explanations of terms or abbreviations may be found or described in Boudreau, 2013, Circadian variation of heart rate variability across sleep stages, Sleep 36(12): 1919; Chaudhry, 2020, Sleep in the natural environment, Sensors 20(5): 1378; Iber, 2007, The AASM Manual for the Scoring of Sleep and Associated Events: Rules, Terminology and Technical Specifications, American Academy of Sleep Medicine, Westchester; Korkalainen, 2020, Deep learning enables sleep staging from photoplethysmogram for patients with suspected sleep apnea, Sleep 43(11):zsaa098; Mikkelsen, 2019, Machine-learning-derived sleep-wake staging from around-the-ear electroencephalogram outperforms manual scoring and actigraphy, J Sleep Res 28(2):e12786; Schulz, 2008, Rethinking sleep analysis, J Clin Sleep Med 4(2):99-103; and Stephansen, 2018, Neural network analysis of sleep stages enables efficient diagnosis of narcolepsy, Nat Comm 9:5229, the contents of each of which are incorporated by reference.

What is claimed is:

1. A method for analyzing sleep patterns, the method comprising:
receiving, by a computer system, data for a subject from a first sensor on a first wearable device;
preprocessing the data based on a type of the wearable device into standardized data; and
analyzing the standardized data with the computer system to identify sleep stages at different times for the subject.

2. The method of claim 1, further comprising receiving second data from a second subject using a second wearable device, wherein the data and the second data are output from the respective devices with different formats or different content.

3. The method of claim 2, comprising preprocessing the second data into a format matching that of the standardized data.

4. The method of claim 2, wherein the first wearable device and the second wearable device each include at least one photoplethysmographic (PPG) sensor and at least one accelerometer, and further wherein the first wearable device and the second wearable device output PPG and acceleration data with different formats.

5. The method of claim 1, wherein the computer system includes one or more software modules that interact with each application programming interface (API) associated with a plurality of different wearable devices from different manufacturers.

6. The method of claim 1, further comprising receiving second data from a second subject using a second wearable device, wherein the computer system instructs the second wearable device to turn on an optional mode that captures heart rate at a resolution greater than a default mode for the second wearable device.

7. The method of claim 6, wherein the optional mode is a workout mode.

8. The method of claim 1, wherein the analyzing step involves presenting the standardized data to a machine learning system that assigns a sleep stage to each of a plurality of epochs in the standardized data.

9. The method of claim 8, wherein each sleep stage is selected from the group consisting of wake, REM sleep, and non-REM sleep.

10. The method of claim 8, wherein the machine learning system includes at least one neural network that captures time dependencies and has been trained on labeled training data from multiple subjects.

11. The method of claim 10, wherein the neural network that captures time dependencies is a recurrent neural network.

12. The method of claim 11, wherein the recurrent neural network is a long short-term memory neural network.

13. The method of claim 8, wherein the machine learning system outputs records of sleep stages or intervals for each subject, wherein contents of the records are consistent regardless of the different formats or different content between the data from the wearable device and the second data from the second wearable device.

14. The method of claim 8, wherein the computer system creates a record of sleep stages for the patient, and provides access to the record to a clinician who is a registered user of the computer system.

15. The method of claim 1, wherein the computer system has stored therein a profile for each of a plurality of subjects, each profile identifying a resting heart rate or a heart rate variability threshold for a respective subject.

16. The method of claim 15, wherein the analyzing step includes filtering and smoothing the standardized data, comparing the smoothed data to thresholds from the profile for the subject, merging sleep sequences based on the thresholds, and creating a record with sleep intervals for the subject, wherein the record includes at least one sequence of bedtime, sleep onsite, wakeup, and rise time.

17. A system for analyzing sleep patterns, the system comprising:
a processor coupled to memory containing instructions executable to cause the system to:
receive data for a subject from a first sensor on a first wearable device;
preprocess the data based on a type of the wearable device into standardized data; and
analyze the standardized data to identify sleep stages at different times for the subject.

18. The system of claim 17, further operable to receive second data from a second subject using a second wearable device, wherein the data and the second data are output from the respective devices with different formats or different content.

19. The system of claim 18, further operable to preprocess the second data into a format matching that of the standardized data.

20. The system of claim 18, wherein the first wearable device and the second wearable device each include at least one photoplethysmographic (PPG) sensor and at least one accelerometer, and further wherein the first wearable device and the second wearable device output PPG and acceleration data with different formats.
